(19) United States
(12) Patent Application Publication
(10) Pub. No.: US 2003/0134305 A1
(43) Pub. Date: Jul. 17, 2003

(54) Method for the enumeration of mammalian micronucleated erythrocyte populations with a single-laser flow cytometer

(76) Inventors: Stephen D. Dertinger, Webster, NY (US); Dorothea K. Torous, Rochester, NY (US); Carol R. Tometsko, Rochester, NY (US)

Correspondence Address: Edwin V. Merkel, NIXON PEABODY LLP, Clinton Square, P.O. Box 31051, Rochester, NY, 14603-1051, US

(21) Appl. No.: 10/223,305

(22) Filed: Aug. 16, 2002

Related U.S. Application Data
(60) Provisional application No. 60/312,709, filed on Aug. 16, 2001.

(51) Int. Cl. C12Q 1/68; G01N 33/567; G06F 19/00; G01N 33/48; G01N 33/50

(52) U.S. Cl. 435/6; 435/7.21; 702/21

(57) ABSTRACT

A single-laser flow cytometric method for the enumeration of micronucleated erythrocyte populations is disclosed which affords superior fluorescent resolution of young reticulocytes from normochromatic erythrocytes, particularly in mammals that exhibit efficient splenic sequestering. The method can also be used for assessing the DNA damaging potential of pharmaceuticals undergoing clinical trials; evaluating patient-specific responses to chemotherapy or radiation therapy; evaluating compounds, diets, or other factors that may protect against DNA damage resulting from endogenous or exogenous agents; evaluating compounds, diets, or other factors that may potentiate DNA damage resulting from endogenous or exogenous agents; determining the level of DNA damage in a population following a major accident; and monitoring workers who may be occupationally exposed to DNA damaging agents

This application claims the benefit of U.S. Provisional Patent Application Serial No. 60/312,709 filed Aug. 16, 2001, which is hereby incorporated by reference in its entirety.

The present invention was made, at least in part, using funding received from the National Institutes of Environmental Health Sciences under grant numbers 1R43ES010752-01 and 2R44ES010752-02. The U.S. government may have certain rights in this invention.

### FIELD OF THE INVENTION

The present invention is directed to medical applications, and to the field of toxicology, in which a need exists for a rapid, sensitive and economical method for evaluating chromosome damage. Specifically, the present invention relates to a process for analyzing the frequency of micronuclei in mammalian erythrocyte populations by a rapid and sensitive single-laser flow cytometric method.

### BACKGROUND OF THE INVENTION

Micronuclei (“MN”) are extra-nuclear chromatin resulting from double-strand DNA breaks or from mitotic spindle apparatus dysfunction. Since MN are the result of clastogenic or aneugenic activity, researchers in academia, industry and government have utilized in vivo rodent micronucleus assays to screen chemical and physical agents for clastogenic and aneugenic activity (Hayashi et al., “In vivo Rodent Erythrocyte Micronucleus Assay,” Mutat. Res. 312:293-304 (1994)). When these studies are performed with mice, micronuclei are often scored in peripheral blood erythrocyte populations, as these cells persist in peripheral circulation. Whereas micronucleated erythrocytes persist in mice, they are actively sequestered and eliminated from circulation by the spleen of most other mammalian species, including the rat and human. For this reason, micronucleus studies that involve mammalian species other than the mouse have tended to measure the incidence of MN in newly formed erythrocytes obtained from the bone marrow compartment (that is, before spleen sequestering). Aside from bone marrow aspirates, MN induction in human erythrocytes has also been studied in peripheral blood, but a focus has been on splenectomized subjects. Given the removal of MN-containing erythrocytes from the peripheral blood of humans and most other mammals, it had been assumed that the peripheral blood compartment of eusplenic individuals would not ordinarily provide a sensitive indication of clastogenic or aneugenic exposure (Schlegel et al., “Assessment of Cytogenetic Damage by Quantitation of Micronuclei in Human Peripheral Blood Erythrocytes,” Cancer Res. 46:3717-3721 (1986)).

The requirement for either bone marrow aspirates or blood from splenectomized volunteers has tended to limit the widespread study of micronucleus formation in human erythrocyte populations. Even so, some data are available from these cohorts, and they demonstrate the sensitivity of the endpoint for evaluating cytogenetic damage resulting from chemo- or radiotherapy, and for investigating associations between nutritional or lifestyle factors and cytogenetic damage (Krogh Jensen et al., “Cytogenetic Effect of Methotrexate on Human Cells in vivo,” Mutat. Res. 64:339-343 (1979); Abe et al., “Micronuclei in Human Bone Marrow Cells: Evaluation of the Micronucleus Test Using Human Leukemia Patients Treated with Antileukemic Agents,” Mutat. Res. 130:113-120 (1984); Smith et al., “Micronucleated Erythrocytes as an Index of Cytogenetic Damage in Humans: Demographic and Dietary Factors Associated with Micronucleated Erythrocytes in Splenectomized Subjects,” Cancer Res. 50:5049-5054 (1990); Zuniga et al., “Micronucleated Erythrocytes in Splenectomized Patients With and Without Chemotherapy,” Mutat. Res. 361:107-112 (1996)).

More recently, there have been examples of genotoxicant-induced increases in MN in the peripheral blood of non-splenectomized subjects, so long as analyses are restricted to newly formed erythrocytes (i.e., young reticulocytes, or RETs). For example, Abramsson-Zetterberg et al. have described a method whereby human blood erythrocytes are applied to paramagnetic beads coated with an antibody specific for the transferrin receptor, which is also known as CD71 (Human Cytogenetic Biomonitoring Using Flow-cytometric Analysis of Micronuclei in Transferrin-positive Immature Peripheral Blood Reticulocytes,” Environ. Molec. Mutagen 36:22-31 (2000)). Newly formed erythrocytes express the CD71 antigen on their surface, while mature erythrocytes do not. Therefore, RETs bind to these antibody-coated beads while mature erythrocytes, which are also known as normochromatic erythrocytes (“NCEs”), do not. A powerful magnet is used to retain the bead-bound cells in a vessel while unbound cells are eluted. An enzymatic process is used to release bead-associated cells, and the resulting blood fraction is thereby enriched for immature erythrocytes. This fraction, now enriched for CD71-positive erythrocytes (RETC^{CD71+}) is then fixed and stained with thiazole orange and Hoechst dyes as these investigators have previously described for rodent samples (Grawe et al., “Flow-cytometric Enumeration of Micronucleated Polychromatic Erythrocytes in Mouse Peripheral Blood,” Cytometry 13:750-758 (1992)). The sophisticated and lengthy cell processing steps and requirement for a dual-laser flow cytometer may limit the adoption of this procedure by other laboratories. Nevertheless, this data set demonstrates several important points. First, when MN are measured in the RET^{CD71+} cell population of healthy volunteers, micronucleus frequencies approximate those observed in the bone marrow compartment. This suggests that the effect of spleen sequestering is minimized when analyses are restricted to RET^{CD71+}. Secondly, subjects undergoing cancer therapy with DNA damaging agents exhibit a time-dependent increase in micronucleated CD71-positive reticulocytes (MN-RET^{CD71+}). These data are consistent with the hypothesis that genotoxicant-induced MN-RET^{CD71+} can be detected in the peripheral blood compartment of mammalian species, whether the subjects have an efficient MN-sequestering spleen or not.

A simpler automated system for scoring micronucleated erythrocyte populations of mammals would find many applications that include, but are not limited to: (1) assessing the DNA damaging potential of pharmaceuticals undergoing clinical trials; (2) evaluating patient-specific responses to chemotherapy or radiation therapy; (3) evaluating compounds, diets, or other factors that may either protect against or potentiate DNA damage resulting from endogenous or exogenous agents; (4) determining the level of DNA damage in a population following a major accident; and (5) monitoring workers who may be occupationally exposed to DNA damaging agents.

Accordingly, there is a need for a simple, rapid, and accurate method for enumerating the frequency of micronucleated erythrocyte populations. Such a technique would preferably use fluorescent labels and/or dyes that differentially stain and quantify four erythrocyte sub-populations: mature and immature erythrocytes, with and without micronuclei. Furthermore, a desirable characteristic of these fluorescent labels and/or dyes is that they are all excited by a similar wavelength but each exhibits significantly different emission spectra, thus enabling the use of a single-laser flow cytometer.

The present invention is directed to overcoming the above-noted deficiencies in the art.

### SUMMARY OF THE INVENTION

One aspect of the present invention relates to a single-laser flow cytometric method for the enumeration of micronucleated erythrocyte populations, the method including: providing a fixed sample comprising erythroctye populations including mature normochromatic erythrocytes, reticulocytes, micronucleated normochromatic erythrocytes, micronucleated reticulocytes, or combinations thereof, with the erythrocyte populations being in suspension and substantially free of aggregates, permeable to a nucleic acid dye and RNase, with cell surface markers in a form recognizable by an antibody, and exhibiting substantially low autofluorescence; substantially degrading RNA of reticulocytes in the fixed sample with RNase; contacting the fixed sample with fluorescent labeled antibody, having binding specificity for a surface marker for erythroblasts/reticulocytes and having a fluorescent emission maximum which is about 550 nm or greater; staining cellular DNA with a nucleic acid staining dye in a concentration range detectable by flow cytometry and having a fluorescent emission spectrum which does not substantially overlap with the fluorescent emission spectrum of the fluorescent labeled antibody; exciting the nucleic acid staining dye and the fluorescent label associated with the erythrocyte populations using a focused laser beam of appropriate excitation wavelength for both the nucleic acid staining dye and the fluorescent label to produce fluorescent emission; and detecting the fluorescent emission and light scatter produced by the erythrocyte populations and calculating the number of specific erythrocyte populations in said sample.

Other aspects of the present invention relate to various uses of the flow cytometric method of the present invention. These include: a method of assessing the DNA-damaging potential of a pharmaceutical agent, a method of identifying individuals hypersensitive to a DNA-damaging agent, a method of measuring workplace safety of individuals exposed to suspected DNA-damaging agent(s) in a workplace environment, a method of evaluating the effects of an agent which can modify (i.e., enhance or suppress) endogenous or exogenous-induced DNA damage, a method of evaluating the effects of a diet or a dietary nutrient which can modify endogenous or exogenous-induced DNA damage, and a method of measuring the level of DNA damage following exposure of individual(s) to a DNA damaging agent. Following administration or exposure to an agent or event, the flow cytometric method of the present invention is performed on a peripheral blood or bone marrow sample removed from the individual and fixed following its obtention. By comparing the percentage of micronucleated normochromatic erythrocytes and/or micronucleated reticulocytes in the sample from a baseline micronucleated normochromatic erythrocyte and/or micronucleated reticulocyte value in unexposed (i.e., normal) mammals, it is possible to determine whether any difference between the detected level is a significant deviation from the baseline level. Where such a significant deviation exists, it is possible to determine whether the pharmaceutical agent induces DNA damage and the extent thereof, identify an individual who is hypersensitive to a DNA-damaging agent, determine whether or not a workplace environment (exposed to one or more DNA-damaging agents) is unsafe, evaluate whether an agent can modify (i.e., enhance or suppress) endogenous or exogenous-induced DNA damage, evaluate the effects of a diet or a dietary nutrient which can modify endogenous or exogenous-induced DNA damage, and determine the extent of DNA damage following exposure of an individual to a DNA damaging agent.

The extreme rarity of micronucleated erythrocytes in the peripheral blood of most mammalian species has precluded facile enumeration of these events. A high throughput method based on single-laser flow cytometry is described for scoring the incidence of micronuclei in mature and immature erythrocytes. The method described herein allows for the quantification of micronucleated mature erythrocytes and micronucleated immature reticulocytes in mammalian blood sample preparations using a single-laser flow cytometer. The process is able to analyze millions of mature erythrocytes and thousands of reticulocytes in each blood sample, thereby enhancing the accuracy of the measurements. As a result, the flow cytometric-based micronucleus scoring system of the present invention supplies repeatable and reliable data with technical ease and modest equipment requirements.

### BRIEF DESCRIPTION OF THE DRAWINGS

FIG. 1 is a bivariate graph illustrating the resolution of anti-CD71-PE positive erythrocytes (RETs) from more mature erythrocytes. Note that cells were also stained with SYBR Green I nucleic acid dye. In the absence of RNase treatment, SYBR Green I (x-axis) represents a second fluorescent signal which discriminates RETs from NCEs based on RNA content.

FIGS. 2A-B are histograms of green fluorescence associated with malaria-infected rat blood cells that were fixed, washed, and treated with RNase and SYTOX Green dye at 10⁻⁶ or 10⁻⁵ dilutions, respectively. These histograms illustrate the resolution erythrocytes infected with the malaria parasite P. berghei from uninfected erythrocytes (at origin). At dye concentrations in the range of 10⁻⁵ (FIG. 2B), parasite peaks are well resolved and fluorescent intensity is less sensitive to differences in cell density.

FIG. 3 is a bivariate graph of rat peripheral blood sample fixed, washed and incubated with anti-CD71-PE, RNase and SYTOX Green dye. With this method, four erythrocyte populations are resolved: mature and immature erythrocytes, with and without micronuclei.

FIGS. 4A-B are bivariate graphs illustrating two regions which were used to define a gate that excludes non-specific events/debris from quantitative analysis. As shown in FIG. 4A, a side scatter versus forward scatter region requires events to match size and granularity characteristics of single cells. As shown in FIG. 4B, an anti-GPA-CyChrome versus forward scatter region requires events to label positive for the erythroid cell marker glycophorin A.

FIGS. 5A-B are bivariate graphs of human peripheral blood samples fixed, washed and incubated with anti-CD71-PE, anti-GPA-CyChrome, RNase and SYTOX Green dye. With this method, four erythrocyte populations are resolved: mature and immature erythrocytes, with and without micronuclei. FIG. 5B is a bivariate graph prepared using a sample from a splenectomized subject. A higher incidence of micronucleated normochromatic erythrocytes are evident.

FIGS. 6A-B are bivariate graphs of malaria-infected rat blood used for instrument setup. In FIG. 6A, malaria-infected rat blood was incubated with RNase, anti-human-GPA, SYTOX, and anti-human CD71. Samples prepared in this manner were useful for setting PMT voltages and FL1—% FL2 compensation (this compensation eliminates the red component of a green fluorescence nucleic acid dye). Furthermore, this preparation guided the dimensions of the four analysis regions that are depicted. That is, it was useful for setting the boundary which distinguishes CD71-positive RETs from CD71-negative erythrocytes, and erythrocytes with a micronucleus-like DNA content from those without. In FIG. 6B, malaria-infected rat blood was incubated with RNase, anti-human-GPA, SYTOX, and anti-rat CD71. Samples prepared in this manner served as a positive control which exhibited numerous double-positive events that model the target cells of interest (MN-RET). Further, these samples were valuable for evaluating the FL2—% FL1 compensation setting (this compensation eliminates the green component of the PE label). When set appropriately, parasites exhibit a vertical profile. In this manner, erythrocytes with a similar DNA content exhibit comparable SYTOX or SYBR Green fluorescence, irrespective of their CD71 expression level.

### DESCRIPTION OF THE PREFERRED EMBODIMENTS

The present invention is directed to a method for the enumeration of micronucleated erythrocyte populations using a single-laser flow cytometer.

For purposes of the present invention, “erythrocyte populations” is intended to include populations of mature normochromatic erythrocytes, immature erythrocytes such as erythroblasts and/or reticulocytes, micronucleated normochromatic erythrocytes, micronucleated reticulocytes, and combinations thereof. Samples of erythrocyte populations from mammals can be obtained from either peripheral blood or bone marrow. The erythrocyte populations from any mammal can be analyzed in accordance with the present invention, although preferred mammals include rodents, such as rat and mouse, and primates such as monkeys, chimpanzees, and humans. The method of the present invention is particularly useful when examining peripheral blood from humans, because the human spleen is extremely efficient at removing MN-containing erythrocytes from circulation. Consequently, the rarity of these events makes it desirable that the cell labeling and staining methods are extremely specific and provide intensely fluorescent cells, especially for MN-RET. Unless both of these criteria are met, it is not possible to accurately measure very rare cell populations such as MN-RET in peripheral blood circulation of mammals whose spleen sequesterization function is very efficient. For example, in a preferred embodiment of the present invention, fluorescent resolution of reticulocytes is maximized by using antibodies against erythroblasts or reticulocytes which are conjugated to phycoerythrin, relative to some other fluorochromes (e.g., FITC). This results in better fluorescent resolution of reticulocytes from normochromatic erythrocytes, and minimizes the likelihood that other cell types or sub-cellular or non-cellular debris will register fluorescence intensities similar to target cells of interest (i.e., reticulocytes). Similarly, by utilizing a nucleic acid dye that results in exceptionally high fluorescence yield such as SYTOX Green dye, improved resolution of MN-containing erythrocytes from normal (DNA-deficient) erythrocytes is achieved relative to procedures that utilize more common nucleic acid dyes such as propidium iodide. Again, this ensures accurate enumeration of rare cells such as micronucleus-containing erythrocytes by minimizing the chances that spurious cellular, sub-cellular or non-cellular events exhibit fluorescence intensities that are equivalent to target cells of interest.

As for the source of mammalian erythrocytes, conventional procedures can be utilized to obtain samples. For example, a blood sample can be obtained from the tail vein of rodents after a brief warming period under a heat lamp. Alternately, cardiac puncture may be performed on anesthetized animals. In the case of humans, a finger prick with a lancet or a blood draw via standard venipuncture are convenient sources of erythrocytes. In any case, blood should be collected into an anticoagulant (e.g., EDTA or heparin) to prevent aggregation and clot formation.

Bone marrow samples can also be acquired according to standard procedures. Standard buffers which do not lead to cellular aggregation or clotting should be utilized with bone marrow samples.

Once a blood or bone marrow sample has been obtained, the sample is fixed so as to render the erythrocytes and reticulocytes in suspension and substantially free of aggregates, permeable to a nucleic acid dye and RNase, with cell surface markers intact (i.e., in a form recognizable by appropriate antibodies), and exhibiting substantially low autofluorescence. Fixing is accomplished in alcohol at a temperature of about −40° C. to about −90° C. Briefly, a 100 to 1000 μl aliquot of each blood suspension (e.g., from a syringe and needle or from a pipettor) is delivered forcefully into tubes containing a suitable amount (e.g., about 1 to about 11 ml) of ultracold alcohol. It is preferable that the ultracold alcohol fixative is maintained at about −40° C. to about −90° C., preferably about −70° C. to about −90° C. The alcohol may be a primary alcohol or a secondary alcohol. Suitable primary alcohols include but are not limited to ethanol and methanol. Suitable secondary alcohols include but are not limited to isopropyl alcohol. Of these alcohols, methanol is preferred. Once the samples are fixed, the tubes can be struck sharply or vortexed to break up aggregates. The samples can be stored at about −40° C. to about −90° C., preferably about −70° C. to about −90° C. The samples are preferably stored for at least 24 hours.

Prior to analysis, the cells are diluted out of the fixative with ice cold buffered salt solution. In a preferred embodiment, the buffered salt solution is Hank's Balanced Salt Solution (HBSS), or 0.9% NaCl supplemented with sodium bicarbonate, preferably at about 5.3 mM. The cells are centrifuged under conditions which are effective at maintaining cell structure while removing dissolved solids therefrom. Exemplary centrifugation conditions include about 500× to about 1000×g for 5 minutes. Thereafter, supernatants are decanted and the cell pellets are stored at 4° C. or on ice until analysis. Once cells are washed out of alcohol fixative, it is preferable to stain and analyze them with a flow cytometer within about 3 days, more preferably on the same day that they are washed out of fixative.

Once the cells are washed out of fixative, RNA of the reticulocytes is substantially degraded with RNase so that the only nucleic acid that remains is DNA (i.e., DNA of micronuclei, if present). RNase treatment can be carried out by introducing fixed and washed erythrocyte populations into tubes containing an appropriate amount of an RNase A solution (i.e., ˜20 μg RNase/ml HBSS). Incubations with RNase are preferably carried out at about 4° C. to about 25° C.

Following RNase treatment, nucleic acid dyes are used to stain DNA of micronuclei present in erythrocytes or reticulocytes and fluorescent labeled antibodies directed to specific cell surface markers are used to distinguish erythrocytes generally from other cell types and aggregate materials as well as to distinguish one sub-population from another sub-population within the larger erythrocyte population. Alternatively, RNase treatment and antibody marking of erythroid cells can be carried out simultaneously.

Suitable fluorescent labeled antibodies are those which have binding specificity for a surface marker for erythroblasts or reticulocytes (or both) and have a fluorescent label with a fluorescent emission maximum of about 550 nm or greater. As used herein, “a surface marker for erythroblasts/reticulocytes” means at least one species of a surface antigen present on reticulocytes but absent on mature erythrocytes, thereby enabling erythroblasts and reticulocytes to be distinguished from mature erythrocytes by the presence of this marker. Such markers are known in the art to include, but are not limited to, CD71 (a transferrin receptor). As used herein, “a surface marker for erythroid cells” means at least one species of a surface antigen present on erythroid cells (i.e., both reticulocytes and erythrocytes) but absent on other cell lineages. Such antigens are known to practitioners of the art to be useful for restricting analyses to cells that express these markers, while eliminating other cells and/or debris. Such markers include, without limitation, CD233, CD241, CD235a (Glycophorin A, or GPA), CD235ab (Glycophorin A & B), CD236 (Glycophorin C & D), and CD236R (Glycophorin C).

A number of fluorescent labels are available which have the desired excitation and emission characteristics. As used herein, the term “fluorescent label” means at least one species of a fluorescent molecule that is conjugated or otherwise attached to a monoclonal antibody with binding specificity for a surface marker for erythroblasts/reticulocytes or a surface marker for erythroid cells. Because a single laser flow cytometer is intended to be employed with the present invention, the selected fluorescent label used on the antibodies should accommodate the excitation parameters of the laser employed. While other fluorescent labels known in the art may be useful in the method according to the present invention, fluorescent labels having an excitation wavelength in a range of about 470 to about 505 nm are desirable.

Where multiple antibodies are used to label different erythrocyte sub-populations, then it is desirable to utilize different fluorescent labels on each type of antibody, such that each label has an emission spectrum which does not substantially overlap the emission spectra of other labels. Preferably, each has a sufficiently distinct emission maxima that discriminates itself from other fluorescent labels.

A preferred fluorescent labeled antibody directed to a surface marker for erythroblasts/reticulocytes (i.e., discriminating between RTs and mature erythrocytes) is phycoerythrin-anti-CD71 antibody. The phycoerythrin label is characterized by an emission maxima between about 560 nm to about 600 nm.

Preferred fluorescent labeled antibodies directed to a surface marker for erythroid cells (i.e., discriminating between erythrocytes and other cell types or non-cellular or sub-cellular—debris) a Cy-Chrome or PerCP labeled anti-CD233 antibodies, anti-CD241 antibodies, anti-CD235a antibodies, anti-CD235ab antibodies, anti-CD236 antibodies, and anti-CD236R antibodies, and combinations thereof. Of these, the CyChrome-anti-CD235a antibody is most preferred. Both the Cy-Chrome and PerCP labels have emission maxima which are greater than about 650 nm.

Labeling of erythrocytes with selected fluorescent labeled antibodies is achieved by combining antibody solution with the fixed and washed mammalian blood (or bone marrow) sample under conditions effective to allow antibodies to recognize the cell surface markers. Exemplary conditions include an approximately 30 minute incubation period at about 4° C. Thereafter, cells can be washed using, e.g., buffered saline solution or HBSS.

Suitable nucleic acid dyes are those capable of staining cellular DNA at a concentration range detectable by flow cytometry and which have a fluorescent emission spectrum which does not substantially (i.e., significantly) overlap with the fluorescent emission spectrum of the fluorescent labels used on antibodies. Generally, the fluorescent emission maximum of a nucleic acid dye should be in the range of about 545 nm or less, preferably between about 515 nm to about 545 nm. Suitable dyes include, but are not limited to, SYTO dyes 11-16, 18, and 20-25, BOBO-1 iodide, BO-PRO-1 iodide, YOYO-1 iodide, YO-PRO-1 iodide, TOTO-1 iodide, TO-PRO-1 iodide, SYTOX Green and SYBR Green 1 (all from Molecular Probes, Inc.). Of these, SYTOX Green and SYBR Green 1 are preferred.

Washed antibody-labeled cells can be resuspended with a nucleic acid dye solution (e.g., dilution of dye stock solution in HBSS). Nucleic acid dyes are available from a number of suppliers in crystalline form or as highly concentrated stock solutions. It is important to work with nucleic acid dyes whose cell density and dye concentration parameters are known or otherwise have been optimized through experimentation as described in the Example infra. In a preferred embodiment of the present invention, stock solutions of SYTOX Green or SYBR Green 1 dyes are diluted about 10⁻⁴ to 10⁻⁶. Dye loading should be carried out under conditions effective to allow for dye penetration of the cell and binding of the dye to DNA of nucleated cells as well as any micronuclei that may be present. Exemplary conditions include an approximately 15 minute incubation period at about 4° C.

Thereafter, the treated sample can be subjected to flow cytometric analysis using a suitable flow cytometer having a single focused laser beam with an appropriate emission band to excite the nucleic acid dye(s) and fluorescent labeled antibodies. As cells pass through the focused laser beam, the cells possessing antibody labels exhibit a fluorescent emission maxima characteristic of the fluorescent label associated therewith and cells possessing a micronucleus exhibit a fluorescent emission maxima characteristic of the nucleic acid dye. The flow cytometer is equipped with appropriate detection devices to enable detection of the fluorescent emissions and light scatter produced by the erythrocyte populations. Cells are counted and the number of specific erythrocyte sub-populations in the sample can be calculated.

Prior to such excitation and detection of fluorescence from the treated samples, the single-laser flow cytometer can be calibrated for the detection of micronuclei. This can be achieved using a biological standard which has been treated in parallel with the fixed sample (i.e., RNase, antibody treatment, nucleic acid stain, etc.). Preferred biological standards are fixed erythrocyte samples obtained from a malaria-infected mammal, more preferably a Plasmodium berghei-infected rodent (e.g., rat or mouse). The use of the biological standard mimics the micronucleated erythrocytes. As a result of the use of such biological standards, it is possible to achieve one or more of the following: setting photomultiplier tube voltage, setting electronic compensation parameters, and defining the position of regions that indicate micronucleus-containing erythrocytes.

Further aspects of the present invention relate to the use of the present invention to monitor the effects of DNA damage events on individuals and to monitor the protective effects of various agents on those individuals. In using the method of the present invention to assess damage or monitor protective agents, determinations of such effects are typically based on statistically significant differences as measured using appropriate statistical analyses.

Therefore, the present invention can be used after administering a DNA-damaging agent to a mammal prior to obtaining a blood sample. The administering of the DNA-damaging agent can be performed anywhere from about 1 to about 4 days, preferably about 1 to about 2 days, prior to obtaining the blood sample. To monitor the protective effects of a suspected protective agent, the suspected protective agent can be administered to the individual simultaneous or contemporaneous with administration of the DNA-damaging agent. By contemporaneous, administration of the protective agent is intended to occur before, after, or both before and after administration or exposure to the DNA damaging agent. Preferably, contemporaneous administration occurs within about 12 hours (i.e., before and/or after). Any protective effect afforded by the suspected protective agent can be measured relative to damage caused in the absence of the suspected protective agent or to historical data based on the degree of damage normally afforded by the DNA-damaging agent.

Physical DNA damaging agents that can be tested include, but are not limited to: gamma radiation, beta radiation, and UV radiation. Chemicals which damage DNA that can be tested include, but are not limited to: inorganic genotoxicants (e.g., arsenic, cadmium and nickel), organic genotoxicants (especially those used as antineoplastic drugs, e.g., cyclophosphamide, cisplatin, vinblastine, cytosine arabinoside, etc.), anti-metabolites (especially those used as antineoplastic drugs, e.g., methotrexate and 5-fluorouracil), organic genotoxicants that are generated by combustion processes (e.g., polycyclic aromatic hydrocarbons such as benzo(a)pyrene), as well as organic genotoxicants that are found in nature (e.g., aflatoxins such as aflatoxin B1).

Putative protective agents can be vitamins, bioflavonoids and anti-oxidants, dietary supplements (e.g., herbal supplements), and dietary adjustments (e.g., diets high in beneficial foods and low in processed foods), or any other protective agent, naturally occurring or synthesized by man.

As a result of such monitoring, the present invention can be used to assess the DNA-damaging potential of a pharmaceutical agent by administering a pharmaceutical agent to a mammalian subject and then performing the flow cytometric method analysis of the present invention on a mammalian subject sample, wherein a significant deviation in the percentage of micronucleated NCEs and/or micronucleated RTs from a baseline micronucleated NCE and/or micronucleated RT value in unexposed subject (i.e., placebo-receiving mammalian subject) indicates the genotoxic potential of the pharmaceutical agent. The level of damage can also be assessed. The greater the deviation from the baseline value, the greater the extent or level of damage caused by the pharmaceutical agent.

Likewise, such monitoring can be used to identify individuals that are hypersensitive to a DNA-damaging agent by administering a genotoxic agent to a mammalian subject and then performing the flow cytometric method analysis of the present invention on a subject sample, wherein a significant deviation in the percentage of micronucleated NCEs and/or micronucleated RTs from a baseline micronucleated NCE and/or micronucleated RT value in unexposed mammals indicates the hypersensitivity of the mammalian subject to the genotoxic agent.

Furthermore, as part of a routine protocol following an adverse event in a workplace environment (e.g., radiation leak or carcinogenic agent spill), such monitoring can be used to define the extent of harm a workplace presents as well as the successfulness of a workplace cleanup. These monitoring approaches can be carried out by performing the flow cytometric method of the present invention using samples obtained from mammals exposed to one or more DNA-damaging agents in a workplace environment, wherein a significant deviation in the percentage of micronucleated NCEs and/or micronucleated RTs from a baseline micronucleated NCE and/or micronucleated RT value in unexposed mammals indicates that the workplace contains one or more DNA-damaging agents. In addition, the level of damage caused by such agents to which the mammals were exposed indicates the severity of the workplace contamination.

Because of the interaction of agents, it is possible that certain agents may offer protective benefit while other agents may present a magnified risk when combined. For this reason, the present invention can be used to evaluate the effects of an agent which can modify (i.e., enhance or suppress) endogenous or exogenous-induced DNA damage by administering an agent that may modify endogenous or exogenous-induced genetic damage to a mammalian patient and then performing the flow cytometric method of the present invention on a sample from the patient. A significant deviation in the percentage of micronucleated NCEs and/or micronucleated RTs from a baseline micronucleated NCE and/or micronucleated RT value in unexposed mammals indicates that the agent can modify endogenous or exogenous-induced DNA damage. A reduction in the percentage of micronucleated NCEs and/or micronucleated RTs compared to baseline figures indicates a suppression of DNA-induced damage, whereas an increase in the percentage of micronucleated NCEs and/or micronucleated RTs compared to baseline figures indicates an enhancement of DNA-induced damage.

As noted above, diet and dietary nutrients are one type of potentially protective agents. Thus, another aspect of the invention relates to a method of evaluating the effects of a diet or a dietary nutrient which can modify endogenous or exogenous-induced DNA damage. This can be achieved by subjecting a mammal to a predetermined diet or a dietary nutrient that may modify endogenous or exogenous-induced DNA damage, either with or without exposure to endogenous or exogenous agents that can induced DNA damage. The flow cytometric method of the present invention is performed on samples from the mammal, wherein an insignificant deviation in the percentage of micronucleated NCEs and/or micronucleated RTs from a baseline micronucleated NCE and/or micronucleated RT value in unexposed mammals indicates that the diet can modify endogenous or exogenous-induced DNA damage.

### EXAMPLES

The following examples are provided to illustrate embodiments of the present invention, but they are by no means intended to limit its scope.

### Example 1

### Blood Cell Fixation for Analysis by Flow Cytometry

A suitable and reproducible fixing procedure is needed to provide cells from erythrocyte populations that are compatible with subsequent staining and analysis by flow cytometry. For the present invention, the fixing procedure should provide cells with the following characteristics: (1) predominately in suspension as single cells, (2) permeable to nucleic acid staining dyes and RNase, (3) bearing a CD71 antigen or other surface marker for erythroblast/reticulocyte recognition by a respective antibody, (4) bearing a Glycophorin antigen or other surface marker for erythroid cell recognition by a respective antibody, and (5) low auto fluorescence.

In this example, blood was collected by finger prick from a human volunteer into a tube containing 2 ml heparin solution (500 USP units per ml phosphate buffered saline). Cells were fixed by forcefully injecting 180 μl of the cell suspension into tubes containing 2 ml of various alcohol fixative solutions at 4° C. or −85° C. The alcohol fixative solutions were: methanol, ethanol, isopropyl alcohol, and methanol plus acetic acid mixture (3 parts MeOH, 1 part AA). Immediately after cells were added, the tubes were struck sharply several times and returned to 4° C. or −85° C. overnight. The next day, cells were washed out of fixative by adding 12 ml ice cold HBSS and centrifuged at approximately 1200 RPM for 5 minutes. The supernatants were decanted and the pellets of cells were tapped loose. Cells were observed with a fluorescent microscope after being applied to acridine orange-coated slides (which differentially stains DNA and RNA). Microscopically, the morphology of cells was recorded, and the extent of cellular aggregation and cell recovery were qualitatively evaluated. Cells were also analyzed with a flow cytometer (Becton Dickinson FACSCalibur, 488 mn excitation). For these analyses, 20 μl cell suspension was added to tubes containing 1 ml HBSS with propidium iodide (approximately 1.2 μg/ml). Cell density (i.e., relative cell recovery) was indicated by the number of cells acquired per second, the degree of cellular aggregation was evaluated by measuring the fraction of events that registered a high forward scatter signal, permeability of cells to propidium iodide was noted by measuring the fraction of erythrocytes that exhibited a high FL3 channel signal (presumably RETs), and the relative degree of autofluorescence was indicated by noting the relative FL1 channel signal. These observations have been compiled in Table 1 below. These results indicate that particular advantage is gained by using methanol as fixative, and that ultracold temperatures are most desirable.

TABLE 1
Comparison of Fixatives for Blood Sample Preparation

| Fixative | Temp. | Cell Recovery | Aggregation | PI permeable | Autofluorescence |
|---|---|---|---|---|---|
| Methanol | 4° C. | High | Moderate | Yes | Moderate |
| Methanol | −85° C. | High | Low | Yes | Low |
| Ethanol | 4° C. | High | Moderate | Yes | High |
| Ethanol | −85° C. | High | Moderate | Yes | Low |
| Isopropyl | 4° C. | High | Moderate | Yes | High |
| Isopropyl | −85° C. | Very Low | — | — | — |
| MeOH:AA | 4° C. | Very Low | — | — | — |
| MeOH:AA | −85° C. | Very Low | — | — | — |

### Example 2

### Labeling with Anti-CD71 to Differentially Stain Reticulocytes and Mature Erythrocytes

The ability to differentiate newly formed erythrocytes from more mature erythrocytes is an important component of this invention. Therefore, reagents and processes for differentially labeling these erythrocyte sub-populations were identified. An example whereby useful antibodies for human cell analyses were identified is described below.

20 μl aliquots of a fixed and washed human blood sample were added to flow cytometry (FCM) tubes containing 80 μl anti-CD71 antibody solution. The commercially available anti-CD71 antibodies studied were purchased as FITC or PE conjugates, and were evaluated over a range of concentrations. After incubating for 30 minutes at 4° C., cells were washed with HBSS and resuspended with a nucleic acid dye (propidium iodide or SYBR Green I). In the absence of RNase treatment, these dyes represented a second label independent of CD71 expression that was used to distinguish RETs from NCEs. Cells were analyzed with a flow cytometer (Becton Dickinson FACSCalibur, 488 nm excitation).

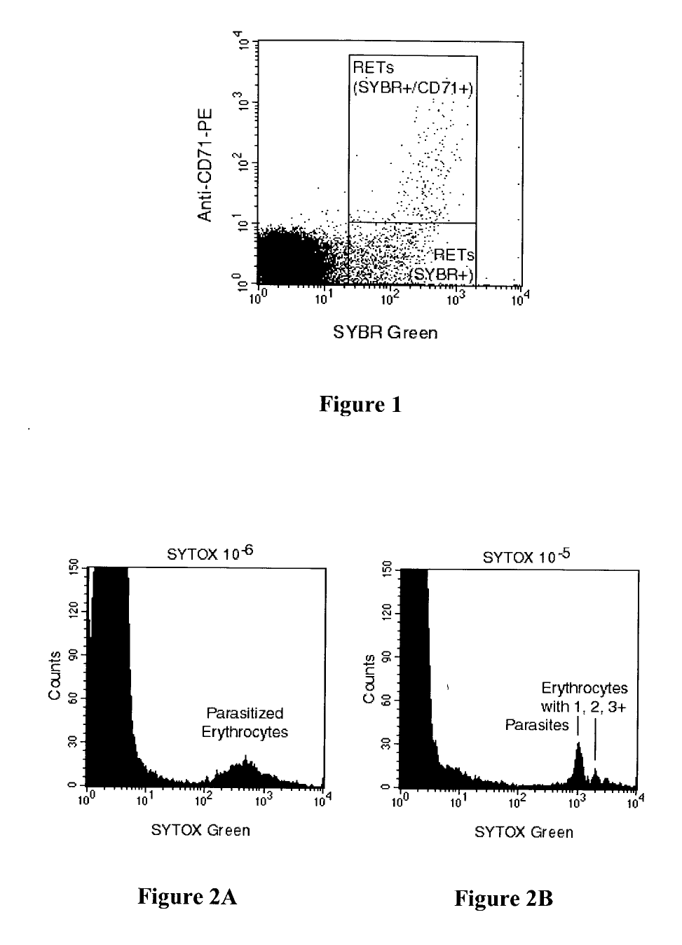

Commercially available fluorescent anti-CD71 antibodies were able to differentially label RETs. Clones that provided sufficient fluorescent resolution are designated L01.1 and M-A712 and Ber-T. Approximately 2 logs of fluorescent resolution was achieved with the PE-conjugates (FIG. 1). For maximizing fluorescent resolution of RETs and NCEs, PE appears to be particularly advantageous. Each PE molecule contains up to 40 fluorescent bile pigments (phycobilins), and these pigments are naturally arranged so that there is minimal fluorescence quenching. After conjugation to antibodies, there is little additional quenching, resulting in conjugates with high quantum yield compared to other fluorophore-antibody conjugates such as FITC.

An important feature of this preferred embodiment of the invention is that it is the very youngest fraction of reticulocytes which stain positive for the erythroblast/reticulocyte label. This is evident from FIG. 1, which demonstrates that it is the most immature RETs (that is, those with the highest RNA-associated fluorescence signal) that are positive for CD71. There is an advantage to differentially labeling the most immature fraction of RETs and enumerating MN in this age cohort. This is expected to minimize the impact that spleen sequestering has on MN frequencies.

### Example 3

### Nucleic Acid Dyes for Flow Cytometric Measurement of Micronuclei

The ability to differentiate erythrocytes with and without MN is an important component of this invention. As such, reagents and processes for differentially labeling these erythrocyte sub-populations were determined. An example whereby useful nucleic acid dyes for mammalian cell analyses were identified is described below.

Nucleic acid dyes that were expected to be compatible with single-laser flow cytometry and an erythroblast/reticulocyte label were examined. Nucleic acid dyes were evaluated with fixed malaria-infected rat blood. Malaria infects the target cells of interest (erythrocytes), endowing them with an MN-like DNA content. Whereas MN are rare and heterogeneous, malaria parasites are abundant and uniform in DNA content. These cells provided an effective means for evaluating potential nucleic acid dyes for the micronucleus scoring application. In these experiments, 20 μl aliquots of fixed and washed malaria-infected rat blood were added to FCM tubes containing 80 μl RNase A solution (20 μg RNase/ml HBSS). Following successive 30 minute incubations at 4° C. and about 25° C., 1 ml ice-cold HBSS containing a range of nucleic acid dye concentrations was added. The dyes evaluated in this manner were: SYTO11, SYTO12, SYTO13, BOBO, YOYO, TOTO, SYTOX Green and SYBR Green I (all from Molecular Probes, Inc.). Dye loading was conducted for at least 15 minutes at 4° C., after which cells were analyzed with a flow cytometer (Becton Dickinson FACSCalibur, 488 nm excitation).

To varying extents, each of these dyes was able to differentially stain erythrocytes with and without malaria parasites for flow cytometric interrogation. SYTOX Green or SYBR Green I dyes were found to be most advantageous. In combination with RNase treatment, parasitized cells are well resolved from uninfected erythrocytes, even at high dilution factors of SYTOX Green or SYBR Green I (10⁻⁵ to 10⁻⁶ dilution of stock solutions provided by Molecular Probes). While 10⁻⁶ dilution of these dyes result in fluorescent separation of parasitized cells, higher dye concentrations (in the range of 10⁻⁵ dilution) demonstrate superior staining characteristics. At these higher concentrations, malaria parasite peaks are consistently tighter (that is, lower coefficients of variance). These features are illustrated in FIGS. 2A-B. Furthermore, at dye concentrations in the range of 10⁻⁵, greater latitude in terms of cell density was observed. That is, malaria peaks were observed to shift to lower FL1 peak channel values as cell densities were increased in solutions of low dye concentration. In contrast, the peak fluorescent channel of malaria-infected cells remained stable over a wide range of cell densities when higher dye concentrations were used.

### Example 4

### Scoring Micronucleated Erythrocyte Populations with One Immunochemical Reagent and One Nucleic Acid Dye

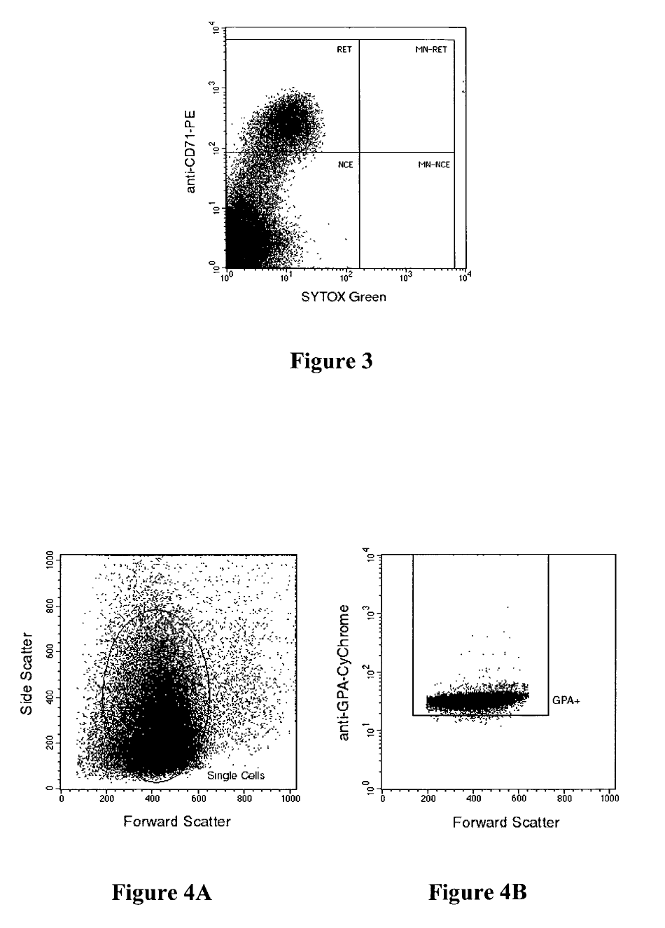

By combining a fluorescent antibody with binding specificity for erythroblasts/reticulocytes plus a nucleic acid dye and RNase, it is possible to quantify the incidence of micronuclei in mammalian erythrocyte populations. In this example, a male rat was treated for 6 days via oral lavage with water and another with cyclophosphamide, a known chromosome-breaking agent (10 mg/kg body weight/day). Approximately 24 hours after the sixth and final administration, blood was collected from the tail vein after a brief warming period under a heat lamp. Blood was maintained in a sodium heparin solution (500 USP units heparin/ml PBS) at room temperature until samples were fixed with −80° C. methanol. The fixed blood samples were divided into several aliquots and were coded for single-blind analysis. After at least 24 hours storage at −80° C. freezer, three aliquots of fixed blood from the water-treated rat and three aliquots of fixed blood from the cyclophosphamide-treated rat were washed out of fixative with 12 ml HBSS. Pellets were tapped loose and 40 μl of each cell suspension was transferred to tubes containing 100 μl labeling solution (1000 μl HBSS with 0.5% fetal bovine serum plus 5 μl anti-rat CD71-PE plus 20 μg RNase A). Cells were then incubated for 30 minutes at 4° C. and then 45 minutes at 37° C. Unbound antibody was washed by adding 3 ml HBSS plus 0.5% fetal bovine serum and centrifuging for 5 minutes at about 600×g. Supernatants were aspirated, and cell pellets were tapped loose. Cells were resuspended with 1 ml HBSS containing 10⁻⁵ dilution of SYTOX Green nucleic acid dye. Sufficient time was allowed for the nucleic acid dye to load and equilibrate with the cells (about 15 minutes at 4° C.). Cells labeled with anti-CD71-PE and stained with SYTOX Green were analyzed with a FACSCalibur flow cytometer (488 nm excitation). The frequencies of RET^{CD71+}, MN-RET^{CD71+} and MN-NCEs were determined. As commonly practiced by flow cytometry operators, events were gated on light scatter characteristics, and in this way subcellular debris or cellular aggregates were excluded from analysis. As illustrated in FIG. 3, the fluorescence emission of the anti-CD71-PE reagent was detected by the FL2 detector, and SYTOX Green fluorescence was detected by the FL1 detector. For these analyses, the stop mode was the acquisition of 20,000 RETs per replicate. The resulting data demonstrate that in this embodiment of the present invention, cyclophosphamide's DNA-breaking activity and cytostatic effects are clearly and consistently detected (as indicated by increased MN-RET^{CD71+} and decreased RET^{CD71+} frequencies, respectively). The raw data from this analysis is provided in Table 2 below.

TABLE 2
Flow Cytometry Measurements for Water and Cyclophosphamide-Treated Rats

| Rat | Treatment | Replicate | % RET^{CD71+} | % MN-NCE | % MN-RET^{CD71+} |
|---|---|---|---|---|---|
| 1 | water | 1 | 4.85 | 0.01 | 0.07 |
| | water | 2 | 4.69 | 0.01 | 0.06 |
| | water | 3 | 4.73 | 0.01 | 0.09 |
| | | Avg. | 4.76 | 0.01 | 0.07 |
| 2 | cyclophosphamide | 1 | 2.57 | 0.04 | 0.63 |
| | cyclophosphamide | 2 | 2.66 | 0.05 | 0.73 |
| | cyclophosphamide | 3 | 2.72 | 0.04 | 0.64 |
| | | Avg. | 2.65 | 0.04 | 0.67 |

RET^{CD71+} = CD71-positive reticulocytes;
MN-NCE = micronucleated normochromatic erythrocytes;
MN-RET^{CD71+} = micronucleated CD71-positive reticulocytes

### Example 5

### Scoring Micronucleated Erythrocyte Populations with Two Immunochemical Reagents and One Nucleic Acid Dye

In a further embodiment of the present invention, a second fluorescent antibody with specificity for erythroid cells is incorporated into the staining procedure. This reagent may be advantageous when analyzing rare events such as micronucleated cells, since more leverage for excluding debris or other sub-cellular particles for analysis is achieved.

Arm venipuncture was employed to collect approximately 1 to 3 ml of blood from ten healthy adult human volunteers as well as three splenectomized but otherwise healthy human volunteers. The blood cells (collected into standard heparin-coated tubes) were transferred to tubes containing 2.5 ml sodium heparin solution (500 USP units/ml PBS). 500 μl aliquots of heparinized blood was fixed by forcefully injecting it into tubes containing 5 ml methanol (−80° C.). After at least 24 hours at −80° C., 1 ml of each fixed and washed cell suspension was transferred to tubes containing 12 ml HBSS. Cells were centrifuged at about 600×g and supernatants were decanted. Pellets were tapped loose and 40 μl aliquots were then incubated with anti-CD71-PE, SYTOX Green, and anti-GPA-CyChrome reagents. Specifically, a 20 μl aliquot of each cell suspension was added to an FCM tube containing 80 μl labeling solution (900 μl HBSS plus 100 μl anti-human CD71-PE plus 1 μl anti-human GPA-CyChrome plus 20 μg RNase A). Cells were held in this solution for 30 minutes at 4° C. followed by 30 minutes at about 25° C. After adding 3 ml HBSS cells were centrifuged for 5 minutes at about 600×g. Cells were resuspended with 1 ml HBSS containing a 10⁻⁵ dilution of SYTOX Green dye. Cells labeled with anti-CD71-PE and anti-GPA-CyChrome, and stained with SYTOX Green were analyzed with a FACSCalibur flow cytometer (488 nm excitation). A region based on light scatter characteristics was drawn on a side scatter versus forward scatter bivariate plot. A region based on GPA-associated fluorescence was drawn on a FL3 fluorescence height versus forward scatter bivariate plot. A gate that combined these two regions was defined so that in order for events to be interrogated for micronucleli, they must exhibit light scatter characteristics of a single cell and stain positive with the anti-GPA-CyChrome reagent. These results are illustrated in FIGS. 4A-B.

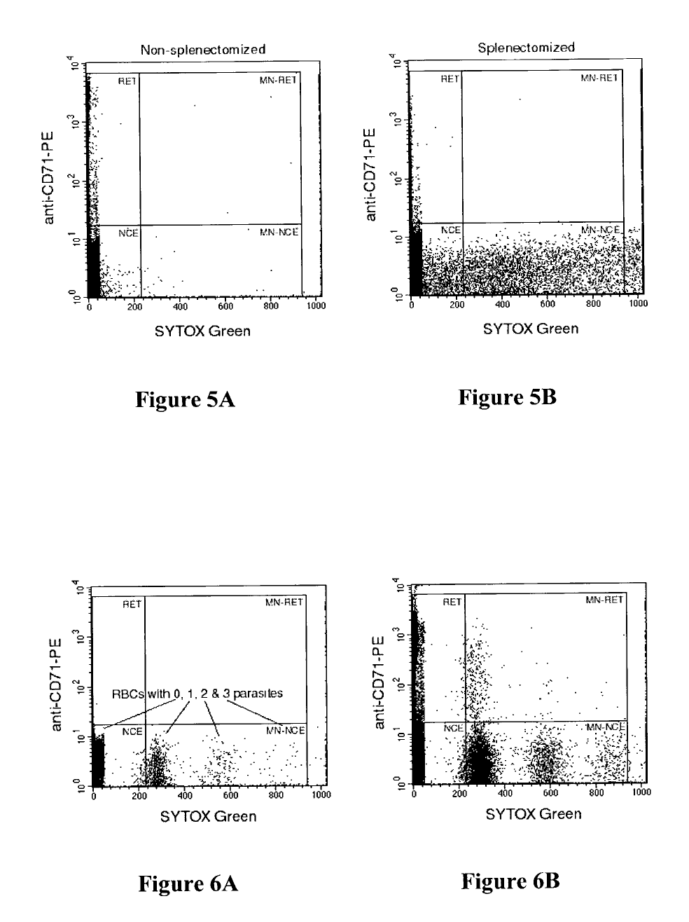

Events which fell within the light scatter and GPA-positive regions were plotted on a FL1 versus FL2 bivariate plot, and the frequencies RET^{CD71+}, MN-RET^{CD71+} and MN-NCE were determined (FIGS. 5A-B). Total RET values for healthy subjects typically range from 1-2%. The majority of the human samples studied herein exhibited RET^{CD71+} frequencies between 0.1 and 0.2% (see Table 3 below). Comparison of these numbers suggests that it is approximately the youngest 10 to 20% of RETs that label with anti-CD71. Presumably, the measurement of micronucleated erythrocyte frequencies in a very immature cell cohort such as this is desirable. This would be expected to minimize the effect that spleen sequestering has on MN-frequency.

TABLE 3
Flow Cytometric Evaluation of Human Blood Samples

| Subject | Sex | Age | Splenectomy? | % RET^{CD71+} | % MN-NCE | % MN-RET^{CD71+} |
|---|---|---|---|---|---|---|
| 1 | F | 34 | No | 0.16 | 0.01 | 0.04 |
| 2 | F | 36 | No | 0.15 | 0.02 | 0.20 |
| 3 | F | 47 | No | 0.26 | 0.03 | 0.12 |
| 4 | F | 51 | No | 0.19 | 0.01 | 0.14 |
| 5 | M | 23 | No | 0.15 | 0.01 | 0.35 |
| 6 | F | 50 | No | 0.09 | 0.02 | 0.34 |
| 7 | F | 23 | No | 0.32 | 0.01 | 0.14 |
| 8 | F | 47 | No | 0.13 | 0.02 | 0.28 |
| 9 | M | 31 | No | 0.17 | 0.01 | 0.16 |
| 10 | M | 42 | No | 0.27 | 0.02 | 0.09 |
| 11 | M | 68 | Yes | 0.36 | 0.03 | 0.18 |
| 12 | M | 45 | Yes | 0.13 | 0.18 | 0.24 |
| 13 | F | 36 | Yes | 0.77 | 0.12 | 0.16 |

RET^{CD71+} = CD71-positive reticulocytes;
MN-NCE = micronucleated normochromatic erythrocytes;
MN-RET^{CD71+} = micronucleated CD71-positive reticulocytes

Given the life-span of the erythrocyte populations that were measured, the observed relationship between % MN-NCE and % MN-RET^{CD71+} was expected. That is, a consistently higher incidence of MN was observed in RET^{CD71+} compared to that in NCEs. In fact, the frequencies of MN-RET^{CD71+} approximate those found in the bone marrow of adults (Krogh Jensen et al., “Cytogenetic Effect of Methotrexate on Human Cells in vivo,” Mutat. Res. 64:339-343 (1979); Abe et al., “Micronuclei in Human Bone Marrow Cells: Evaluation of the Micronucleus Test Using Human Leukemia Patients Treated with Antileukemic Agents,” Mutat. Res. 130:113-120 (1984), each of which is hereby incorporated by reference in its entirety). This suggests that methods of the present invention allow scoring of very young cells whose MN frequency has not been greatly affected by splenic filtration. This is further supported by the fact that the splenectomized individuals evaluated had elevated MN-NCE frequencies compared to those for non-splenectomized individuals. These subjects would be expected to exhibit MN-NCE values that more closely approach MN-RET^{CD71+} frequencies since only partial or no spleen function remains. Compared to subjects 12 and 13, subject 11 exhibited a relatively low MN-NCE value. Whereas subjects 12 and 13 were confirmed to have had their spleens removed for non-trauma reasons, subject 11 was splenectomized for unknown reason(s). It is likely that a common occurrence in trauma patients, namely splenosis (i.e., regenerative splenic tissue) explains this somewhat lower MN-NCE value (Pearson et al., “The born-again spleen: return of splenic function after splenectomy for trauma,” N. Engl. J. Med. 298:1389-1392 (1978), which is hereby incorporated by reference in its entirety).

### Example 6

### Calibrating Instrumentation Parameters with a Biological Standard

In a further embodiment of the present invention, biological standards are incorporated into the flow cytometry-based scoring system. This reagent may be advantageous for enumerating micronucleated erythrocyte populations, particularly in regards to consistently and precisely adjusting flow cytometer instrumentation parameters, and for helping to approximate light scatter and fluorescence characteristics of MN-containing mammalian erythrocytes. An example of this embodiment which employs Plasmodium berghei (malaria) infected rat blood erythrocytes as a biological standard is given below.

Arm venipuncture was employed to collect approximately 1 to 3 ml of blood from two healthy adult volunteers. The blood cells (collected into standard heparin-coated tubes) were transferred to tubes containing 2.5 ml sodium heparin solution (500 USP units/ml PBS). 500 μl aliquots of heparinized blood was fixed by forcefully injecting it into tubes containing 5 ml methanol (−80° C.). On five separate days of flow cytometric analysis, 1 ml of fixed cell suspensions was transferred to tubes containing 12 ml HBSS. Cells were centrifuged at about 600×g and supernatants were decanted. Pellets were tapped loose and 20 μl aliquots were then incubated with anti-CD71-PE, SYTOX Green, and anti-GPA-CyChrome reagents. Specifically, 20 μl was added to tubes containing 80 μl labeling solution (900 μl HBSS plus 100 μl anti-human CD71-PE plus 1 μl anti-human GPA-CyChrome plus 20 μg RNase A). Cells were held in this solution for 30 minutes at 4° C. followed by 30 minutes at about 25° C. In a preferred embodiment of the present invention, cells are held for approximately 15 to 30 minutes at 4° C. followed by approximately 30 to 60 minutes at 25° C. to 37° C. After adding 3 ml HBSS cells were centrifuged for 5 minutes at about 600×g. Cells were resuspended with 1 ml HBSS containing a 10⁻⁵ dilution of SYTOX Green dye and allowed to equilibrate for about 15 minutes at 4° C.

Before analyzing the human blood samples, flow cytometry instrumentation and acquisition/analysis software parameters were calibrated based on the fluorescence of malaria-infected rat blood. Two malaria-infected rat blood samples which had been fixed according to methods of the present invention were washed, labeled, and stained in parallel with the human samples on each day of analysis. One aliquot of fixed rodent blood was treated with the exact reagents used for human samples. That is, cells were incubated with RNase, anti-human-CD71-PE and anti-human-GPA-CyChrome. After incubation and washing steps, cells were resuspended with SYTOX Green. This sample 1) guided PMT voltage settings to optimally resolve parasitized (MN-like) erythrocytes, 2) guided FL2—% FL1 compensation setting, 3) guided positioning of quadrant demarcation which delineated erythrocytes with and without MN, and 4) guided positioning of quadrant demarcation which delineated CD71-negative erythrocytes from CD-71 positive erythrocytes (see FIGS. 6A-B).

A second aliquot of malaria-infected blood was similarly stained, but anti-rat-CD71 was substituted for anti-human-CD71-PE. This substitution provided a sample which exhibited CD71-PE and SYTOX Green positive events (i.e., malaria-infected reticulocytes). These events model MN-RETs, the primary endpoint of this scoring system, and were important when considering FL1—% FL2 compensation. Appropriate FL1—% FL2 compensation was evident when parasites exhibited a vertical SYTOX Green fluorescent signal (i.e., consistent DNA-associated fluorescence, irrespective of their CD71 expression level) (see FIGS. 6A-B).

Cells labeled with anti-CD71-PE and anti-GPA-CyChrome, and stained with SYTOX Green as described above were analyzed with a FACSCalibur flow cytometer (488 nm excitation). A region based on light scatter characteristics was drawn on a side scatter versus forward scatter bivariate plot. A region based on GPA-associated fluorescence was drawn on a FL3 fluorescence height versus forward scatter bivariate plot. A gate that combined these two regions was defined so that in order for events to be interrogated for micronuclei, they must exhibit light scatter characteristics of a single cell and stain positive with the anti-GPA-CyChrome reagent.

Events which fell within the light scatter and GPA-positive regions were plotted on an FL1 versus FL2 bivariate plot, and the frequency of RET^{CD71+}, MN-RET^{CD71+} and MN-NCE were determined. As illustrated by Table 4, the reproducibility of these three cell population measurements suggests that inter-experimental variation is low and well controlled when instrumentation parameters are guided by biological standards. Thus, micronucleated erythrocyte populations are measured subsequent to the calibration of flow cytometry instrumentation parameters using biological standards.

TABLE 4
Reproducibility of Flow Cytometric Micronucleus Data

| | Analysis | | | |
|---|---|---|---|---|
| Subject ID | Day | % RET^{CD71+} | % MN-NCE | % MN-RET^{CD71+} |
| 7 | 1 | 0.30 | 0.03 | 0.14 |
| | 2 | 0.32 | 0.01 | 0.14 |
| | 3 | 0.36 | 0.01 | 0.08 |
| | 4 | 0.32 | 0.01 | 0.10 |
| | 5 | 0.34 | 0.01 | 0.09 |
| | Avg. | 0.33 | 0.01 | 0.11 |
| | Stand. Dev. | 0.02 | 0.01 | 0.03 |
| 10 | 1 | 0.24 | 0.02 | 0.11 |
| | 2 | 0.27 | 0.01 | 0.09 |
| | 3 | 0.25 | 0.02 | 0.10 |
| | 4 | 0.29 | 0.01 | 0.10 |
| | 5 | 0.28 | 0.01 | 0.11 |
| | Avg. | 0.27 | 0.01 | 0.10 |
| | Stand. Dev. | 0.02 | 0.01 | 0.01 |

RET^{CD71+} = CD71-positive reticulocytes;
MN-NCE = micronucleated normochromatic erythrocytes;
MN-RET^{CD71+} = micronucleated CD71-positive reticulocytes

### Example 7

### Measuring Patient-Specific Response to Cancer Therapy

Fixed human blood cells collected from a cancer patient prior to and over the course of radiation treatment (total body irradiation) were stained with anti-CD71-PE, SYTOX Green, and anti-GPA-CyChrome. Arm venipuncture was employed to collect approximately 1 to 3 ml of blood from the patient. The blood cells (collected into standard heparin-coated tubes) were transferred to tubes containing 2.5 ml sodium heparin solution (500 USP units/ml PBS). 500 μl aliquots of heparinized blood was fixed by forcefully injecting it into tubes containing 5 ml methanol (−80° C.). On the day of flow cytometric analysis, 1 ml of fixed cell suspensions were transferred to tubes containing 12 ml HBSS. Cells were centrifuged at about 600×g and supernatants were decanted. Pellets were tapped loose and 40 μl aliquots were then incubated with anti-CD71-PE, SYTOX Green, and anti-GPA-CyChrome reagents. Specifically, 40 μl was added to tubes containing 100 μl labeling solution (900 μl HBSS with 0.5% fetal bovine serum plus 100 μl anti-human CD71-PE plus 1 μl anti-human GPA-CyChrome plus 20 μg RNase A). Cells were held in this solution for 30 minutes at 4° C. followed by 30 minutes at about 25° C. In a preferred embodiment of the present invention, cells are held for approximately 15 to 30 minutes at 4° C. followed by approximately 30 to 60 minutes at 25° C. to 37° C. After adding 3 ml HBSS+0.5% fetal bovine serum cells were centrifuged for 5 minutes at about 600×g. Cells were resuspended with 1 ml HBSS containing a 10⁻⁵ dilution of SYTOX Green dye and allowed to equilibrate for about 15 minutes at 4° C.

Cells labeled with anti-CD71-PE and anti-GPA-CyChrome, and stained with SYTOX Green were analyzed with a FACSCalibur flow cytometer (488 nm excitation). A region based on light scatter characteristics was drawn on a side scatter versus forward scatter bivariate plot. A region based on GPA-associated fluorescence was drawn on a FL3 fluorescence height versus forward scatter bivariate plot. A gate that combined these two regions was defined so that in order for events to be interrogated for micronuclei, they exhibited light scatter characteristics of a single cell and stained positive with the anti-GPA-CyChrome reagent.

Events which fell within the light scatter and GPA-positive regions were plotted on an FL1 versus FL2 bivariate plot, and the frequency of RET^{CD71+}, MN-RET^{CD71+} and MN-NCE were determined. As illustrated by Table 5 below, the patient exhibited a treatment-related increase in MN-RET^{CD71+} frequency. The effect of irradiation is not evident at the second time point, since it was collected less than 12 hours after initiation of treatment. This is expected, since RET formed during and subsequent to irradiation treatment in the bone marrow compartment would not begin to enter peripheral blood circulation until approximately 16 to 24 hours has elapsed. As for the return to baseline levels, this too is expected, since the last blood draw was obtained approximately one month after completion of treatment.

TABLE 5
Treatment-Related Increase in Micronucleus Frequency

| Blood | Accumulated | | | |
|---|---|---|---|---|
| Draw | Dose (Gy) | % RET^{CD71+} | % MN-NCE | % MN-RET^{CD71+} |
| First | 0 | 0.34 | 0.01 | 0.11 |
| Second | 1.5 | 0.30 | <0.01 | 0.05 |
| Third | 10.5 | 0.01 | <0.01 | 2.14 |
| Fourth* | 12 | 0.19 | 0.01 | 0.13 |

RET^{CD71+} = CD71-positive reticulocytes;
MN-NCE = micronucleated normochromatic erythrocytes;
MN-RET^{CD71+} = micronucleated CD71-positive reticulocytes;
*Fourth blood sample collected approximately one month after completion of treatment

In summary, the efficiency by which most mammalian species' spleen removes MN-containing erythrocytes from circulation has been considered a severe obstacle to utilizing peripheral blood MN-RET measurements as a reliable indicator of cytogenetic damage in eusplenic individuals (Schlegel et al., “Assessment of Cytogenetic Damage by Quantitation of Micronuclei in Human Peripheral Blood Erythrocytes,” Cancer Res. 46:3717-3721 (1986), which is hereby incorporated by reference in its entirety). While bone marrow donors and splenectomized subjects have been studied effectively, the limit on the available pool of subjects has prevented widespread use of this endpoint. The data presented herein suggest that it may be possible to evaluate cytogenetic damage in the form of micronuclei in the peripheral blood of non-splenectomized subjects in addition to those lacking splenic function. This is made possible by the methods of the present invention. These methods enable investigators to restrict MN enumeration to the most immature fraction of RETs, thereby minimizing the effect of spleen function. This scoring system may find many applications in the fields of medicine and toxicology. An incomplete list of applications include: assessing the DNA damaging potential of pharmaceuticals undergoing clinical trials; evaluating patient-specific responses to chemotherapy or radiation therapy; evaluating compounds, diets, or other factors that may protect against DNA damage resulting from endogenous or exogenous agents; evaluating compounds, diets, or other factors that may potentiate DNA damage resulting from endogenous or exogenous agents; determining the level of DNA damage in a population following a major accident; and monitoring workers who may be occupationally exposed to DNA damaging agents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A single-laser flow cytometric method for the enumeration of micronucleated erythrocyte populations, the method comprising:
providing a fixed sample comprising erythrocyte populations including mature normochromatic erythrocytes, reticulocytes, micronucleated normochromatic erythrocytes, micronucleated reticulocytes, or combinations thereof, with the erythrocyte populations being in suspension and substantially free of aggregates, permeable to a nucleic acid dye and RNase, with cell surface markers in a form recognizable by an antibody, and able to exhibit substantially low autofluorescence;
substantially degrading RNA of reticulocytes in the fixed sample with RNase;
contacting the fixed sample with fluorescent labeled antibody, having binding specificity for a surface marker for erythroblasts/reticulocytes and having a fluorescent emission maximum which is about 550 nm or greater;
staining cellular DNA with a nucleic acid staining dye in a concentration range detectable by flow cytometry and having a fluorescent emission spectrum which does not substantially overlap with the fluorescent emission spectrum of the fluorescent labeled antibody;
exciting the nucleic acid staining dye and the fluorescent label associated with the erythrocyte populations using a focused laser beam of appropriate excitation wavelength for both the nucleic acid staining dye and the fluorescent label to produce fluorescent emission; and
detecting the fluorescent emission and light scatter produced by the erythrocyte populations and calculating the number of specific erythrocyte populations in said sample.

2. The flow cytometric method according to claim 1 wherein said providing comprises:
obtaining a peripheral blood or bone marrow sample from a mammal; and
fixing the erythrocytes and reticulocytes in the sample in alcohol at a temperature of from about −40° C. to about −90° C.

3. The flow cytometric method according to claim 2 further comprising:
administering a DNA-damaging agent to the mammal prior to said obtaining.

4. The flow cytometric method according to claim 3 further comprising:
administering a suspected protective agent that protects against DNA damage to the mammal simultaneous or contemporaneous with administration of the DNA-damaging agent; and
measuring any protective effect afforded by the suspected protective agent.

5. The flow cytometric method according to claim 2 wherein the sample is from peripheral blood.

6. The flow cytometric method according to claim 2 wherein the sample is from bone marrow.

7. The flow cytometric method according to claim 2 wherein the alcohol is a primary alcohol.

8. The flow cytometric method according to claim 7 wherein the primary alcohol is ethanol or methanol.

9. The flow cytometric method according to claim 2 wherein the alcohol is a secondary alcohol.

10. The flow cytometric method according to claim 9 wherein the secondary alcohol is isopropyl alcohol.

11. The flow cytometric method according to claim 1 further comprising:
removing unbound fluorescent labeled antibody from the treated sample.

12. The flow cytometric method according to claim 11 wherein said removing comprises:
washing the treated sample and
exposing the washed sample to centrifugal forces sufficient to separate unbound fluorescent labeled antibody from fluorescent labeled antibody bound to cells.

13. The flow cytometric method according to claim 11 wherein said removing is carried out prior to said staining with nucleic acid dye.

14. The flow cytometric method according to claim 1 wherein the fluorescent labeled antibody having binding specificity for a surface marker for erythroblasts/reticulocytes is Phycoerythrin-anti-CD71 antibody.

15. The flow cytometric method according to claim 1 wherein the fluorescent labeled antibody having binding specificity for a surface marker for erythroblasts/reticulocytes has a fluorescent emission maximum which is between about 560 nm and about 600 nm.

16. The flow cytometric method according to claim 1, wherein the nucleic acid staining dye is a dye having a fluorescent emission maximum in the range of about 545 nm or less.

17. The flow cytometric method according to claim 16 wherein the nucleic acid staining dye is a dye having a fluorescent emission in the range of about 515 nm to about 545 nm.

18. The flow cytometric method according to claim 16 wherein the nucleic acid staining dye is a dye selected from the group consisting of SYTO dyes 11-16, 18, and 20-25, BOBO-1 iodide, BO-PRO-1 iodide, YOYO-1 iodide, YO-PRO-1 iodide, TOTO-1 iodide, TO-PRO-1 iodide, SYTOX Green and SYBR Green I.

19. The flow cytometric method according to claim 1 wherein said exciting and detecting are carried out using a single laser flow cytometer.

20. The flow cytometric method according to claim 19 further comprising:
calibrating the single laser flow cytometer using a biological standard which has been treated in parallel with the fixed sample.

21. The flow cytometric method according to claim 20 wherein said calibrating comprises setting photomultiplier tube voltage, setting electronic compensation parameters, defining the position of regions that indicate micronucleus-containing erythrocytes, and combinations thereof.

22. The flow cytometric method according to claim 20 wherein the biological standard is a fixed erythrocyte sample obtained from a malaria-infected mammal.

23. The flow cytometric method according to claim 22 wherein the malaria-infected mammal is a Plasmodium berghei-infected rodent.

24. The flow cytometric method according to claim 1 wherein said contacting further comprises:
contacting the cells with a second fluorescent labeled antibody having binding specificity for a surface marker for erythroid cells and having a fluorescent emission which does not substantially overlap with the fluorescent emission of the fluorescent labeled antibody or the fluorescent emission of the nucleic acid staining dye.

25. The flow cytometric method according to claim 24 wherein the second fluorescent labeled antibody comprises a CyChrome label.

26. The flow cytometric method according to claim 24 wherein the second fluorescent labeled antibody has a fluorescent emission maximum in a range of about 650 nm or greater.

27. The flow cytometric method according to claim 24 wherein the second fluorescent labeled antibody is selected from the group consisting of fluorescent labeled anti-CD233 antibodies, anti-CD241 antibodies, anti-CD235a antibodies, anti-CD235ab antibodies, anti-CD236 antibodies, and anti-CD236R antibodies, and combinations thereof.

28. The flow cytometric method according to claim 24 wherein the second fluorescent labeled antibody is a CyChrome-anti-CD235a antibody.

29. The flow cytometric method according to claim 1 wherein said substantially degrading and said contacting are carried out simultaneously.

30. A method of assessing the DNA-damaging potential of a pharmaceutical agent comprising:
administering a pharmaceutical agent to a mammalian subject and
performing the flow cytometric method according to claim 1 on a peripheral blood or bone marrow sample of the mammalian subject, wherein a significant deviation in the percentage of micronucleated normochromatic erythrocytes and/or micronucleated reticulocytes from a baseline micronucleated normochromatic erythrocyte and/or micronucleated reticulocyte value in unexposed mammals indicates the genotoxic potential of the pharmaceutical agent.

31. A method of identifying individuals hypersensitive to a DNA-damaging agent comprising:
administering a DNA-damaging agent to a mammalian subject; and
performing the flow cytometric method according to claim 1 on a peripheral blood or bone marrow sample of the mammalian subject, wherein a significant deviation in the percentage of micronucleated normochromatic erythrocytes and/or micronucleated reticulocytes from a baseline micronucleated normochromatic erythrocyte and/or micronucleated reticulocyte value in unexposed mammals indicates the hypersensitivity of the mammalian subject to the DNA-damaging agent.

32. The method according to claim 31 wherein the DNA-damaging agent is a physical DNA damaging agent or a chemical DNA damaging agent.

33. The method according to claim 32 wherein the physical DNA damaging agent is selected from the group of gamma radiation, beta radiation, and UV radiation.

34. The method according to claim 32 wherein the chemical DNA damaging agent is selected from the group of inorganic genotoxicants, organic genotoxicants, and anti-metabolites.

35. A method of measuring workplace safety of individuals exposed to one or more suspected DNA-damaging agents in a workplace environment comprising:
performing the flow cytometric method according to claim 1 using peripheral blood or bone marrow samples obtained from mammals exposed to one or more DNA-damaging agents in a workplace environment, wherein a significant deviation in the percentage of micronucleated normochromatic erythrocytes and/or micronucleated reticulocytes from a baseline micronucleated normochromatic erythrocyte and/or micronucleated reticulocyte value in unexposed mammals indicates that the workplace contains one or more DNA-damaging agents.

36. The method according to claim 35 wherein the one or more suspected DNA-damaging agents are physical DNA damaging agents, chemical DNA damaging agents, or combinations thereof.

37. The method according to claim 36 wherein the physical DNA damaging agent is selected from the group of gamma radiation, beta radiation, and UV radiation.

38. The method according to claim 36 wherein the chemical DNA damaging agent is selected from the group of inorganic genotoxicants, organic genotoxicants, and anti-metabolites.

39. A method of evaluating the effects of an agent which can modify endogenous or exogenous-induced DNA damage comprising:
administering an agent that may modify endogenous or exogenous-induced genetic damage to a mammalian subject; and
performing the flow cytometric method according to claim 1 on a peripheral blood or bone marrow sample of the mammalian subject, wherein a significant deviation in the percentage of micronucleated normochromatic erythrocytes and/or micronucleated reticulocytes from a baseline micronucleated normochromatic erythrocyte and/or micronucleated reticulocyte value in unexposed mammals indicates that the agent can modify endogenous or exogenous-induced DNA damage.

40. The method according to claim 39 wherein said administering is carried out simultaneously or contemporaneously with endogenously induced DNA damage.

41. The method according to claim 39 further comprising:
exposing the mammalian subject to an exogenous DNA-damaging agent.

42. The method according to claim 41 wherein said administering is carried out simultaneously or contemporaneously with said exposing.

43. A method of evaluating the effects of a diet or a dietary nutrient which can modify endogenous or exogenous-induced DNA damage comprising:
subjecting a mammal to a predetermined diet or a dietary nutrient that may modify endogenous or exogenous-induced DNA damage; and
performing the flow cytometric method according to claim 1 on a peripheral blood or bone marrow sample of the mammal, wherein an insignificant deviation in the percentage of micronucleated normochromatic erythrocytes and/or micronucleated reticulocytes from a baseline micronucleated normochromatic erythrocyte and/or micronucleated reticulocyte value in unexposed mammals indicates that the diet can modify endogenous or exogenous-induced DNA damage.

44. The method according to claim 43 wherein said subjecting is carried out simultaneously or contemporaneously with endogenously induced DNA damage.

45. The method according to claim 43 further comprising:
exposing the mammal to an exogenous DNA damaging agent.

46. The method according to claim 45 wherein said subjecting is carried out simultaneously or contemporaneously with said exposing.

47. A method of measuring the level of DNA damage following exposure of individual(s) to a DNA damaging agent comprising:
performing the flow cytometric method according to claim 1 on a peripheral blood or bone marrow sample of a mammal exposed to a DNA damaging agent, wherein a significant deviation in the percentage of micronucleated normochromatic erythrocytes and/or micronucleated reticulocytes from a baseline micronucleated normochromatic erythrocyte and/or micronucleated reticulocyte value in unexposed mammals indicates that the agent caused DNA damage and wherein greater deviation from the normal percentage indicates the level of the DNA damage.

48. The method according to claim 47 wherein the DNA-damaging agent is a physical DNA damaging agent or a chemical DNA damaging agent.

49. The method according to claim 48 wherein the physical DNA damaging agent is selected from the group of gamma radiation, beta radiation, and UV radiation.

50. The method according to claim 48 wherein the chemical DNA damaging agent is selected from the group of inorganic genotoxicants, organic genotoxicants, and anti-metabolites.
